(19) United States
(12) Patent Application Publication
(10) Pub. No.: US 2015/0065918 A1
(43) Pub. Date: Mar. 5, 2015

(54) PLACENTAL BLOOD EXTRACTION DEVICE

(71) Applicant: SG MediTech Pte Ltd., Singapore (SG)

(72) Inventors: Tiong Han Toh, Singapore (SG); Ron Wright, Singapore (SG); Gabriel Tan, Singapore (SG); Saey Tuan Barnabas Ho, Singapore (SG); Mari Krizia Ong Leal, Singapore (SG)

(21) Appl. No.: 14/375,089

(22) PCT Filed: Jan. 28, 2013
(86) PCT No.: PCT/SG2013/000035

§ 371 Date: Jul. 28, 2014

(30) Foreign Application Priority Data
Jan. 27, 2012 (SG) 201200701-9

(51) Int. Cl. A61B 5/15; A61B 5/157

(52) U.S. Cl. CPC A61B 5/150045; A61B 5/150068; A61B 5/150083; A61B 5/157; A61B 5/150992; A61B 5/150038; A61B 5/150358; A61B 5/150755; A61B 5/150099; A61B 5/150076; A61B 5/150366; A61B 5/15003; A61B 5/150229
USPC 600/581; 600/573; 600/584

(57) ABSTRACT

A placental blood extraction device comprises a receptacle for receiving a placenta with umbilical cord, the receptacle comprising a compartment arranged to receive the placenta; and a tube extending from the compartment for receiving the umbilical cord; wherein the receptacle is selectively removable from the device for receiving the placenta. The placental blood extraction device may comprise a pressure application device for applying pressure to a placenta, the pressure application device comprising a plurality of pressing members for applying pressure on a plurality of regions of the placenta.

### FIELD OF TECHNOLOGY

The present invention relates to a device and method for extracting and/or collecting placental blood.

### BACKGROUND

It is well known that umbilical cord blood (UCB) is an increasingly important and rich source of stem cells. It is known that stem cells can divide to create new red blood cells which carry oxygen to the brain, new white blood cells used in the body's immune system and new platelets which can assist in blood clotting. It is currently estimated that stem cells may be used for the treatment of over 45 malignant and non-malignant diseases. Such diseases may include certain cancers such as leukaemia, immune and genetic disorders.

UCB may also provide a readily available source of stem cells for transplantation in many situations where bone marrow is currently used. Hence, the use of UCB instead of other sources of stem cells such as for example bone marrow and peripheral blood has many advantages. Such may include for example the reduction or elimination of risk involved in the collection of UCB.

UCB is also easier to collect and harvest while avoiding the risks associated with general anesthesia, which is required for the purposes of extracting bone marrow. UCB is also readily available when needed, assuming an efficient and systematic collection and storage procedure. It has been found that UCB is also more often compatible with people undergoing transplants. Furthermore UCB has a lower procurement cost. It has also been demonstrated that UCB has broader potential clinical applications for improving neural repair, bone and tissue growth.

As such, the importance of UCB is now widely recognized. Blood centres worldwide may collect and store UCB after delivery of a baby subject to the parents' consent or request.

However, a problem associated with UCB is that its collection appears to be a one time possibility and the amount of blood that can be collected is limited using current blood collection technology. Such current blood collection technology may include syringe assisted and gravity assisted methods.

A conventional placental blood extraction device include a chamber for receiving a placenta and pressure application mechanism for applying a pressure to the surface of the placenta to assist extracting as much placental blood as possible.

However, it has been particularly cumbersome to place a placenta properly into the chamber, and particular caution must be taken to prevent the device from being contaminated by the placenta before a second one is placed into the device. Cross contamination between placentas must also be avoided. A known method of preventing contamination is to provide a disposable membrane around the internal surface of the chamber before placing the placenta.

Further, the device needs to be sterilized between two successive extraction processes. This takes more time and manpower to prepare the device after a first extraction process and before a second extraction and collection process.

### SUMMARY OF INVENTION

In a first aspect, the invention provides a placental blood extraction device comprising a receptacle for receiving a placenta with umbilical cord, the receptacle comprising a compartment arranged to receive the placenta; and a tube extending from the compartment for receiving the umbilical cord; wherein the receptacle is selectively removable from the device for receiving the placenta.

This allows a user to enclose the placenta in a disposable receptacle so as to allow the extraction of cord blood while avoiding direct exposure of biological tissue to the pressure applicator. This facilitates the operation of the device because only minimal sterilization procedures, or no sterilization procedures, are required to ready the device for the next set of cord blood collection.

The placental blood extraction device may further include a frame having wheels said frame having a bracket for suspending the receptacle.

The placental blood extraction device may further include a perfusion system mounted to said frame, said perfusion system including an perfusate bag and a peristaltic pump for pumping perfusate into the placenta. The perfusion system may include a warming device for warming the perfusate bag. The perfusion system may include a warming device for warming blood in the conduit for transferring the perfusant.

The placental blood extraction device may further include a bracket for supporting a blood bag for receiving extracted blood. The blood bag bracket may include a rocking mechanism for rocking the bracket. The blood bag may include sensors for detecting the volume of blood in said bag. The sensor may be a load cell for measuring the weight of the bag.

In a second aspect, the invention provides a placental blood extraction device comprising a pressure application device for applying pressure to a placenta, the pressure application device comprising a plurality of pressing members for applying pressure on a plurality of regions of the placenta.

The device allows application of spatially differential pressure on the placenta to achieve a high efficacy. A uniform or centrically high pressure would hamper the drainage of blood from the placenta to the umbilical cord. This can be averted by a gradual build up of pressure from the periphery of the placenta to the centre where the umbilical cord is attached.

An advantage that may become available as a result of the invention is the ability to drive blood from the periphery of the placenta to the “centre” so as to be extracted from the cord. Whilst centrally located blood is easily extractable, in order to maximize yield, it is necessary to be able to extract blood from the peripheral edge of the placenta. This is then driven towards the cord. A linear analogy is extracting toothpaste from a toothpaste tube. Being a thixotropic material, the lack of flow is not dissimilar to extracting placental blood from the blood vessels in the placenta. As with the toothpaste analogy, to maximize yield, it is necessary to apply pressure at a distal region from the extraction point, and be able to progressively apply pressure towards the extraction to drive the blood to the cord.

It will be appreciated that as placentas are almost never uniform, and will have the cord placed at different locations within the placenta, the term “central” refers to the extraction point of the blood within the compartment. That is, the “central” location is the position of the cord, with the intent being to drive blood towards the cord for extraction.

Having pressing members that are capable of applying peripheral pressure, and may also progressively apply pressure, so as to direct the blood may be an important aspect of at least one embodiment of the present invention.

It will be appreciated that, in some circumstances, maximum yield may not necessarily be the best strategy. With a possible window of opportunity of 15 minutes from delivery of the placenta to clotting or contamination of the blood, being able to extract as much blood as possible within the available time is important. Accordingly, if there is a delay in being able to process the placenta, this window may be dramatically reduced, possibly to the extent that insufficient time is available to maximize yield, but only time to extract whatever is available within the reduced time frame. To this end, in a further embodiment, the pressing members may also be switchable from a rhythmic, variable pressure condition, to aggressively apply pressure to the placenta.

In a further embodiment, the invention may accommodate asymmetric placentas. It will be appreciated that placentas will almost certainly never be uniform in shape. Not only will the placenta be non-uniform, but the position of the cord will vary also.

To this end, in a further embodiment, the pressing members may be subjected to control of a range of parameters, including, but not limited to:
i) Rate, applied pressure and displacement of the pressing members;
ii) Selective operation of individual or groups of pressing members;
iii) Said selective operation providing the peripheral to central driving of blood in the veins of the placenta;
iv) Said selective operation providing different rhythmic cycles for the movement and pressure application by the pressing members;
v) Selective operation to identify and accommodate asymmetry of the placenta and/or position of the cord.

To this end, and in particular for the initialization procedure, the device may include a control system, which may further incorporate adaptive control to adapt operation to varying shapes, size and other parameters of said placentas.

In a third aspect, the invention provides a receptacle for use with a placental blood extraction device, the receptacle comprising a compartment arranged to receive the placenta; and a tube extending from the compartment for receiving the umbilical cord; wherein the receptacle is selectively removable from the device for receiving the placenta.

In a fourth aspect, the invention provides a method of extracting blood from a placenta, comprising the steps of: encapsulating the placenta with umbilical cord using a receptacle; inserting a cannula into the umbilical cord; actuating a placenta blood extracting apparatus with the receptacle loaded therein to apply a pressure to the placenta; and collecting blood flowing from the placenta into the cannula.

The method may further comprise loading the empty receptacle into a placental blood extracting device prior to encapsulation.

The receptacle may comprise a tube extending from a compartment for receiving the umbilical cord, the encapsulation step including the step of clipping an end of the umbilical cord using a weight and allowing the end to fall into the tube.

The method may further comprise perfusing a medium into the cord and placenta.

The medium may include aqueous solutions, anti-coagulant and/or chemicals which aid content retrieval from the placenta and cord.

The actuating step may include the step of applying vibration to the placenta.

The method may further comprise applying a negative pressure to the cannula to facilitate the collection of blood.

In a fifth aspect, the invention provides an umbilical cord cutting device comprising: a first and second portion, coupled at respective ends, in relative rotational engagement about a common axis, each portion having a slot parallel to the common axis; a first rotational position aligning the slots of said portions; a second rotational position misaligning said slots cutting edges on said respective ends; wherein the device is arranged to receive an umbilical cord whilst in the first position and on rotation to the second position the device is arranged to cut the umbilical cord placed therein.

The cutting device may further include a pair of clamps each respectively positioned adjacent to said respective ends, such that the device is arranged to relatively rotate the first and second portions from the first position to a third position whereby said clamps forced into engagement with the cord.

In a sixth aspect, the invention provides a cannula assembly comprising: a catheter; a housing mounted to a portion of said catheter such that a length of catheter projects from said housing; an introducer in sliding engagement with said housing and arranged coaxially with said catheter wherein the introducer is movable from an extended position having a penetration end contiguous with an end of said catheter and a retracted position such that said catheter end projects from said penetration end.

The cannula assembly may further include a selectively releasable lock arranged to lock the introducer in the retracted position. The introducer may be a concentric sleeve positioned externally to the catheter. The retracted position may place the introducer fully retractable within the housing.

The cannula assembly may be arranged for penetrating a vein of an umbilical cord, said catheter having a diameter equal to a diameter of the cord vein.

In a seventh aspect, the invention provides a blood bag assembly comprising: a blood transfer conduit; a blood bag coupled at a first end of said conduit; a cannula coupled at an opposed end of said conduit; a blood sampling site for obtaining a blood sample coupled at a position intermediate said ends; a tapping for an perfusion system coupled at a position intermediate said ends.

The blood bag assembly may further include a perfusion system coupled to the perfusate tapping. The perfusion system may include a perfusate bag. The perfusion system may include a tapping for a peristaltic pump. The perfusion system may include a peristaltic pump coupled to the tapping. The perfusion system may include a warming device for warming the perfusate bag.

### BRIEF DESCRIPTION OF DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 is a perspective view of a placental blood extracting device, a receptacle and a blood bag, according to a first embodiment of the invention.

FIG. 2 is an exploded view of the receptacle of FIG. 1.

FIG. 3A is a perspective view of a receptacle according to a second embodiment of the invention and a cannula.

FIG. 3B is a perspective view of the receptacle of FIG. 3A loaded with a placenta with the cannula inserted into the umbilical cord.

FIGS. 4A and 4B illustrate a process of loading a placenta into a receptacle.

FIG. 5A shows a sectional view of a pressure application device according to a third embodiment of the invention.

FIGS. 5B and 5C are perspective view and part cutaway view of an arrangement of the pressing members of the pressure application device.

FIGS. 6A and 6B illustrate two further exemplary constructions of a pressure application device.

FIG. 7 is a schematic diagram of a placental blood extracting device.

FIG. 8 is a flow diagram of a placental blood extracting process.

FIGS. 9A and 9B are elevation views of a placenta transfer system according one embodiment of the present invention.

FIG. 9C is an elevation view of a perfusion device for the placenta transfer system of FIG. 9A.

FIGS. 10A to 100 are sequential views of the in utero extraction of cord blood according to one embodiment of the present invention.

FIGS. 11A and 11B are various views of an umbilical cord cutter according to one embodiment of the present invention.

FIGS. 12A to 12D are sequential views of the cutting of an umbilical cord using device according to the present invention.

FIGS. 13A to 13C are elevation views of a cannula according to one embodiment of the present invention.

FIGS. 14A and 14B are comparison schematic views of blood extraction using a cannula assembly according to one embodiment of the present invention compared to a cannula of the prior art.

FIG. 15 is a schematic view of a blood bag assembly according to one embodiment of the present invention.

FIGS. 16A and 16B are schematic views of the blood bag assembly of FIG. 15.

### DETAILED DESCRIPTION OF EMBODIMENTS

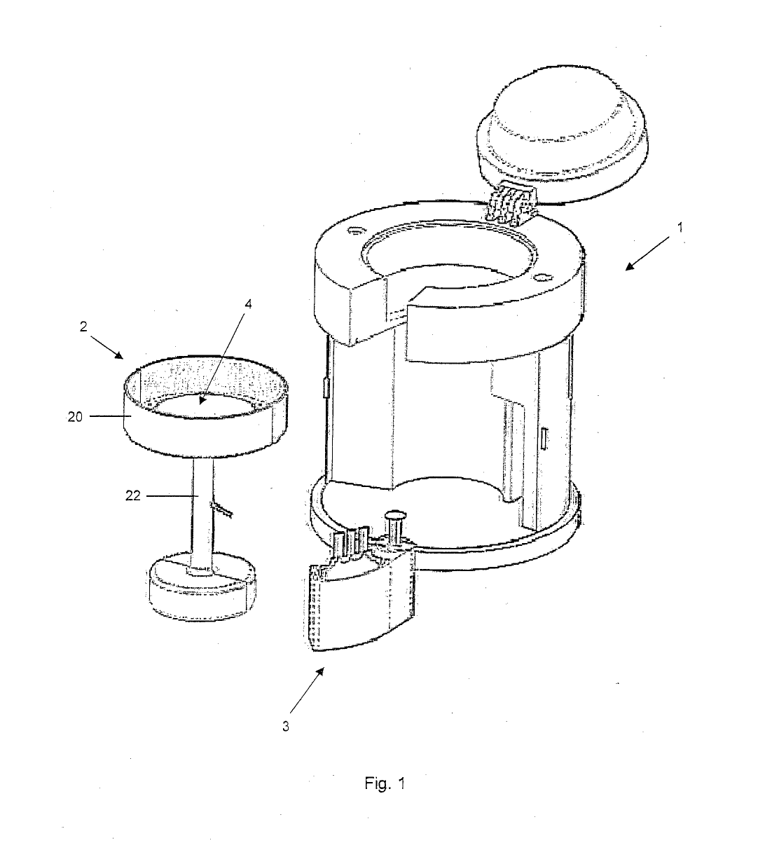

FIG. 1 shows a placental blood extraction device 1, together with a receptacle 2 having a delivered placenta 4 loaded therein and a blood bag 3 for using in the placental blood extracting device 1.

The placental blood extraction device 1 may further comprise a placenta receiving bay for receiving a receptacle having a placenta loaded therein. The placenta 4 may be prepared off-site with the receptacle 2. The blood extracting process can start once the placental blood extracting device 1 is loaded with a receptacle 2, which has been prepared with a placenta 4 preloaded. The placental blood is collected in a blood bag 3.

An exemplary construction of the receptacle 2 is shown in FIG. 2. A delivered placenta 4 normally includes a placenta 40 with an umbilical cord 42 connected. The receptacle 2 may comprise a compartment 20 into which the placenta 40 is placed, and a tube 22 extending from the compartment for receiving the umbilical cord 42. The receptacle 2 is selectively removable from the device 1 for receiving a placenta.

The receptacle may further include a base, having an absorbent material therein for collecting excess blood, and so preventing spillage to maintain a clean and encapsulated collection process. The base may also be conveniently used to allow the receptacle to stand alone, aiding the insertion and removal of the placenta. It will be appreciated that the placenta may remain within the receptacle for disposal of the entire unit, and so also aid in maintain a clean and efficient collection and disposal process. Alternatively, the placenta may be removed for separate disposal, with the receptacle either cleaned and re-used or disposed of separately.

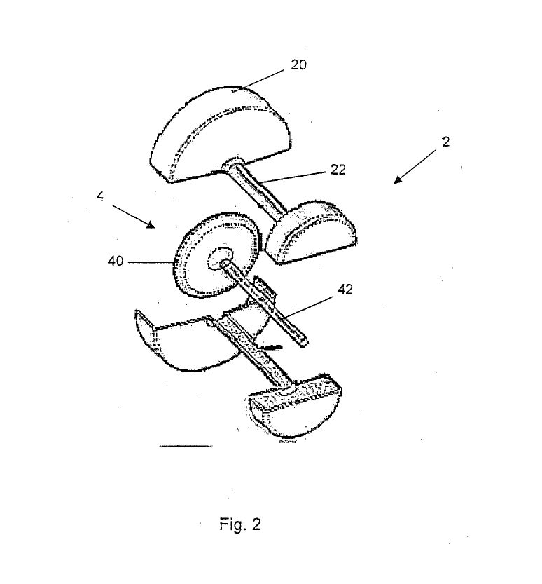

The receptacle 2 may be made of a plurality of components such as two mating parts as shown in FIG. 2, or a single piece as shown in FIGS. 3A and 3B. The compartment 20 according to one embodiment may define a conical or funnel-shaped space of appropriate size suitable for receiving a placenta 40. The apex of the compartment 20 is connected to a tube 22 having a length and cross-sectional area suitable to contain the umbilical cord 42. The other side of the compartment 20 may be an opening allowing pressure to be applied onto the placenta 40.

Optionally, the receptacle 2 may further comprise a cover 24 as shown in FIGS. 3A and 3B. FIG. 3A shows a further embodiment of the receptacle 2 and a cannula 28 for insertion into the umbilical cord through the tube 22. The cover 24 allows for the full encapsulation of the placenta, not merely for the application of pressure, but also to seal the placenta from sources of contamination that may affect the extracted blood. Having the receptacle as a single disposable unit with the placenta sealed therein, ensures the collection process is clean, efficient and avoids direct contact with either the extraction device or the operator.

The receptacle 2 may be made of disposable material, such that after each process a user may simply take out the receptacle 2 and replace it with another one, as if replacing a cartridge.

The compartment 20 may have a circular rim. The compartment 20 and tube 22 may be made of plasticised PVC, so as to be flexible and relatively soft to avoid damage to the placenta, while the circular rim may be made of hard PVC to provide rigidity. It will be appreciated that materials providing similar benefits may be used without departing from the invention.

The tube 22 may be lined with one or more rectangular windows, or recesses 26 to provide users an easy access to the umbilical cord within it. These windows 26 may be covered by flaps to prevent unnecessary exposure of the umbilical cord 42 to the surroundings. Given that umbilical cords vary substantially in length, and that the cord may also be trimmed prior to the extraction process, it is important for the receptacle to allow for this variation by providing multiple cannulation sites. It will be appreciated that, to avoid contamination by maternal blood, a region immediately adjacent to the end of the cord is not available for extracting blood. Nevertheless, by providing multiple cannulation sites, an optimum placement of the cannula is possible after the placenta is placed in the receptacle, providing a convenient and clean setup for the extraction process.

The tube 22 may be formed of suitable material, shape and/or dimension to facilitate the gripping of the umbilical cord 42. Further, the tube 22 may be made of stiff material to prevent it from being penetrated by a needle or cannula to be inserted into the umbilical cord 42 so as to prevent a possible “needle stick” injury to the user by the needle.

Once a placenta is loaded into the receptacle 2, a cannula 28 may be inserted into the umbilical cord 42 through the windows 26 as shown in FIG. 3B. The cover 24 can be closed. The receptacle 2 is now ready to be loaded into the placental blood extraction device 1.

At least a part of the inner surfaces of the receptacle 2 may be lined with absorbent material and this serves to absorb maternal blood from the placenta 40 and umbilical cord 42.

The cover 24 may comprise a hard plastic rim and a flexible membrane which can be made of silicone or other forms of plastic. After the placenta 4 is inserted into the receptacle 2, the cover 24 can be closed to seal the placenta within the receptacle. The flexible membrane serves to allow easy transfer of pressure from a pressure application device 5 to the placenta.

The receptacle 2 effectively separates the placenta 4 from the placental blood extraction device 1, and thereby preventing the placental blood extraction device 1 being contaminated by the maternal blood or any other fluid on the placenta.

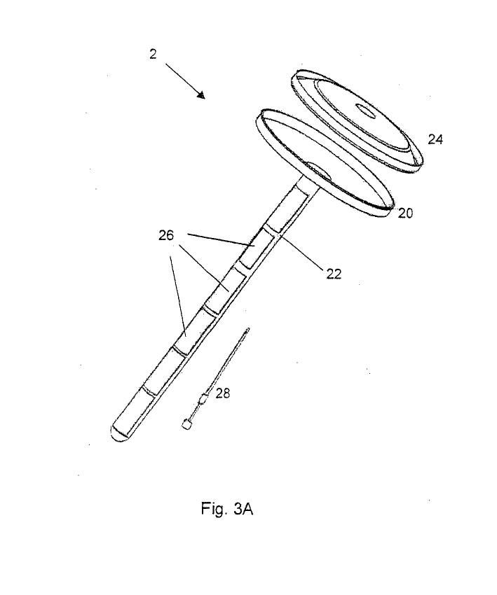

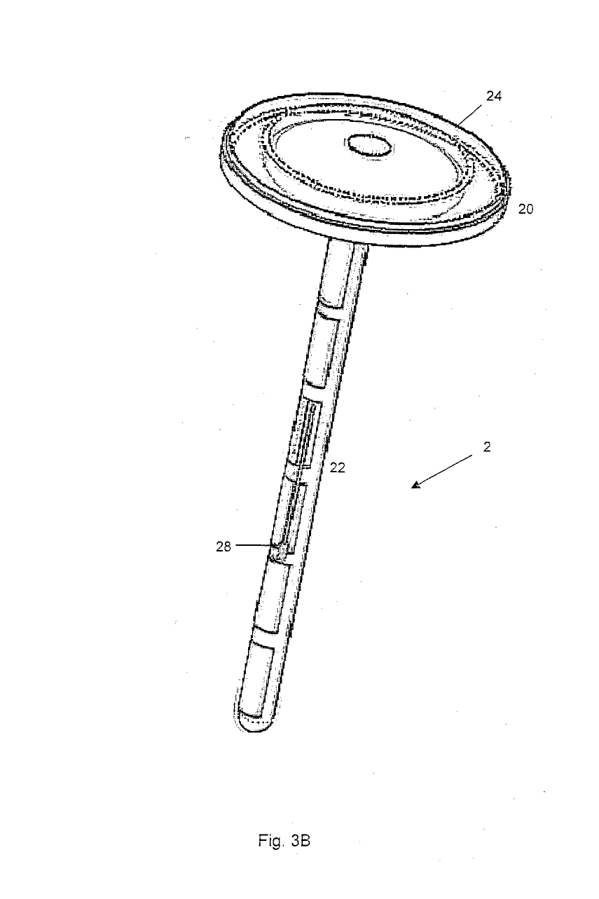

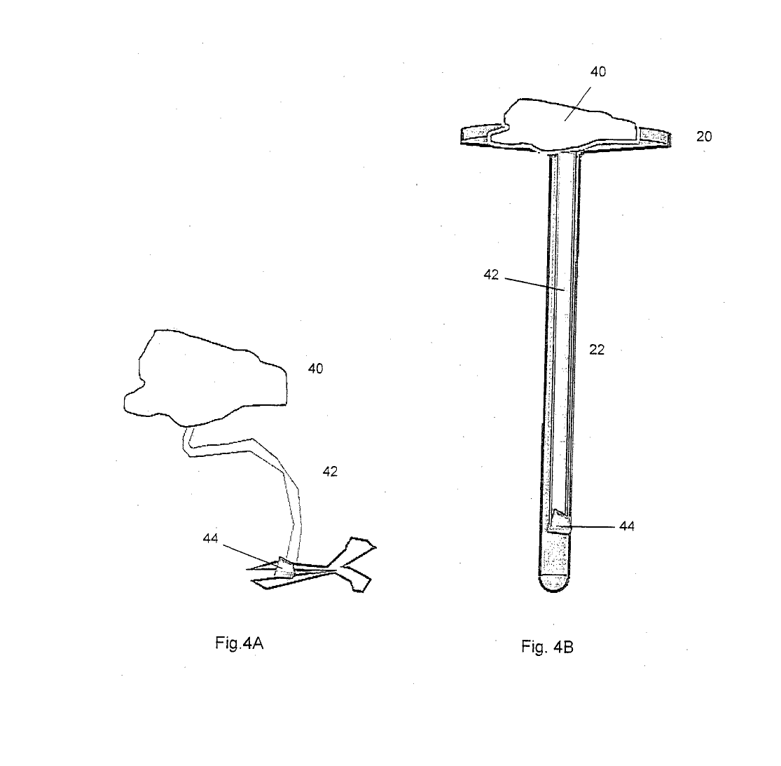

A process of loading a placenta 40 with umbilical cord 42 into a receptacle 2 is shown in FIGS. 4A and 4B. It may include clipping an end of the umbilical cord 42 using a weight 44 and allowing the end to fall into the tube 22.

After the extraction of the placenta and cord from the womb, a weighted clamp 44 will replace the hemostat/clamp that is originally used to clamp the cord during the harvest. It is first clamped at a position on the cord near the hemostat/clamp but on the side nearer to the placenta. Subsequently, the placenta and umbilical cord are inserted into the receptacle, with the cord 42 sliding down the tube 22 and the placenta 40 resting compartment 20. Lastly, the cover 24 is placed onto the compartment 20 to ensure a complete encapsulation of the placenta and cord. The weighted umbilical cord clamp 44 may be specially made heavy in order to allow the umbilical cord 42 to slide easily into the tube 22.

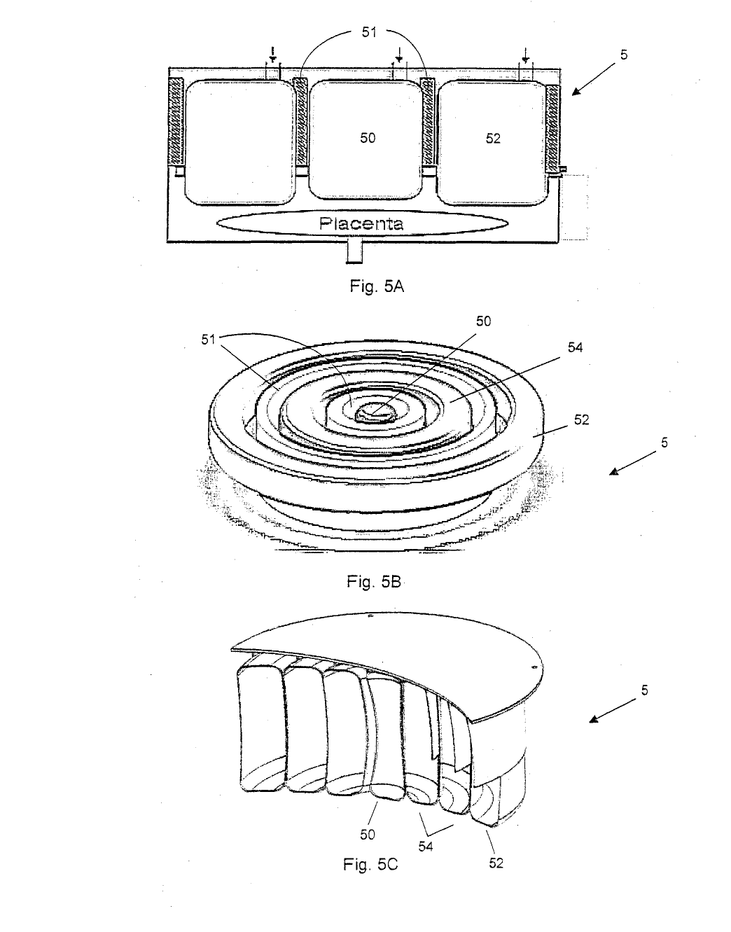

In a further embodiment, the placental blood collection device 1 may comprise a pressure application device 5 for applying pressure to a placenta. FIGS. 5A to 5C are embodiments of a pressure application device 5.

As shown in FIG. 5A, the pressure application device 5 comprises a plurality of pressing members 50, 52 for applying pressure on a plurality of regions of the placenta, wherein the plurality of pressing members 50, 52 are independently controllable so as to allow different levels of pressure to be applied onto different regions of the placenta.

Optionally, at least one of the pressing members 50, 52 may be arranged to apply pressure from the bottom side of the placenta, namely the side of the placenta having umbilical cord extending there from. Accordingly, in this alternative arrangement, it may be possible to apply pressure to the placenta as well as the umbilical as well, optimizing the extraction of blood. The pressure application device 5 allows the timing of activation of the pressure members to be controlled so as to apply a desired rhythm of compression on the placenta.

In one embodiment, the device may further comprise at least one pressure sensor for indicating pressure applied on a region of the placenta. The at least one pressure sensor is attached to at least one of the pressing members. The device may further comprise a control system for controlling the pressing members so as to apply a differential pressure profile on the placenta.

The control system may include a preset algorithm for a differential pressure profile, such as in the form of the rhythmic application of pressure to the placenta. Such algorithm may include a varying operation of the pressing members such that early in the collection process, pressure is applied primarily at the peripheral edges of the placenta, with a central pressure application being of a lesser degree. As the process continues, the pressure may increase to increase the blood yield. The control system may be capable of using a variety of operator selected algorithms.

The control system may further include a pressure sensor for providing feedback signal to the control system. The pressure sensor may be provided on the pressing members. Alternatively, or in addition to, the system may include displacement sensors.

Further, the control system, and/or pressure application device may include a vibratory device to apply vibration to the placenta to further aid the collection process. Such a vibration may or may not be part of the applied algorithm. The control system may be selectively interrupted and/or by-passed by the operator to allow the operator to directly control the rate, magnitude and location of the application of pressure.

The control system may further include an adaptive system. That is, by analyzing signal input from sensors in communication with the control system, the control system may adapt control to meet specific conditions.

For instance, placentas are generally asymmetric, rather than a regular elliptical or circular shape. The control system may include an initialization sequence whereby the shape of the placenta, and position of the umbilical within the placenta may be determined. Such an adaptive system may then adjust the pre-determined arrangement of pressing members to apply pressure according to the determined periphery and cord location. Such an adaptation may include activating certain pressing members that are positioned to contact the placenta, and deactivate other pressing members that do not. Such an arrangement may further redefine pressing members as outer, inner and intermediate pressing members based on the determined shape of the placenta and position of the cord.

The control system may be an open loop system, in which output from sensors are displayed to an operator for their action. Alternatively, the control system may be closed loop, with the control system reacting to sensor input. One such example of a closed loop may include the adaptive system previously described.

At least one of the pressing members, or all pressing members 50, 52, may comprise a resilient portion. Optionally, at least one of the pressing members, or all pressing members 50, 52, may be made wholly of resilient material. The resilient portion or material may prevent potential damage to the placenta during the blood extraction process. The resilient portion or material may include any one of a gel pad, air bag, sponge, or any other suitable material.

In a further embodiment, the plurality of pressing members may comprise at least one outer pressing member 52 for applying pressure onto a peripheral region of the placenta surface. The device may further comprise an inner pressing member 50 for applying pressure onto a central region of the placenta surface.

While loaded in the receptacle or placental blood extraction device, the placenta may resemble roughly an ellipsoid or oblate (flattened) spheroid with a cross-section as shown in FIGS. 6A and 6B. The central region of the placenta surface refers to a region of the surface opposing and/or adjacent to the point from which the umbilical cord extends. This corresponds to the top or bottom surface adjacent to the minor axis. The peripheral region of the placenta surface is the region further away from the minor axis.

The second outer pressing member 52 may be annular, and comprising a pressure application surface for applying pressure on the peripheral region the placenta. It may further comprise one or more intermediate pressing members 54 for applying pressure onto a region between the central and peripheral regions of the placenta surface.

Each of the pressing members 50 and 52 of the pressure application device 5 may be independently controllable to allow different levels of pressure to be applied onto different regions of the placenta. For example, by activating the outer pressing member 52 for a first period, and subsequently activating the inner pressing member 50 for a second period, a differential pressure profile can be applied to the placenta. During the first period, higher pressure is applied on the peripheral region than the central region. During the second period, the pressure applied on the central region increases to expel the blood out of the placenta through the umbilical cord.

In one embodiment, there may be a plurality of outer pressing members 52. Each outer pressing member may have a pressing surface collectively forming an annular shape. The control system may be configured to activate the outer pressing member before activating the mid and/or inner pressing members. The pressing members may be driven by hydraulic means, gearing systems or magnetic force.

Three exemplary types of pressure application device are to be described detail: full air bag compression system, motorized compression system and hybrid compression system.

A full air bag compression system is shown in FIG. 5A to 5C. It may include a plurality of annular inflatable airbags 50, 52 and 54 of various diameters are fitted into a rigid cylindrical structure. These airbags are separated from one another by cylindrical walls 51. During operation, when these air bags are expanded sequentially from the ones on the outer perimeter to the inner ones, the placenta is compressed from the edges towards its centre.

One of the advantages associated with using a full air bag system is that the placenta experiences a gentler compression force as compared to a motorized system involving cams. In addition, it would be more precise in terms of control of the differential pressure across the placenta compared to a single silicone membrane with the dome-shaped cross section.

In a motorized compression system of FIG. 6A, the pressing members are in the form of pads 50, 52 connected to cams 56, or driving members. The cams 56 are capable of reciprocal actuation. These cams 56 will press the pads 50, 52 against the placenta. In order to create a differential pressure across the placenta, the cams 56 can be adjusted to move at different times. For example, if the outer rings of pads 52 may be lowered before the inner rings, such that a differential pressure would be created across the placenta from its perimeter to its center. It will be appreciated that a control system capable of controlling the motorized compression system using preset algorithms as previously described in relation to the air pressure system is also possible. It will be appreciated that a similar system may be used for applying pressure to the umbilical cord, either in combination or as an alternative, so as to extract residual blood. The pressure may be applied to the cord in a systematic manner from the placenta end of the cord to the distal end such that the blood is pushed down the cord to the cannulation site.

A hybrid compression system of FIG. 6B is structurally similar to the motorized compression system except that the pre-inflated airbags 50a and 52a are attached to the pads 50b and 52b, which are in turn connected to the cams 56. This system also has the advantage of a gentler compressive force of the airbags on the placenta. In operation, the cams 56 force the pads 50b and 52b, and the airbags 50a and 52a, downwards onto the placenta and thus compresses the placenta. The outermost ring of the airbag may be compressed first, followed by the inner ones so as to create a differential pressure from its perimeter to its center of the placenta.

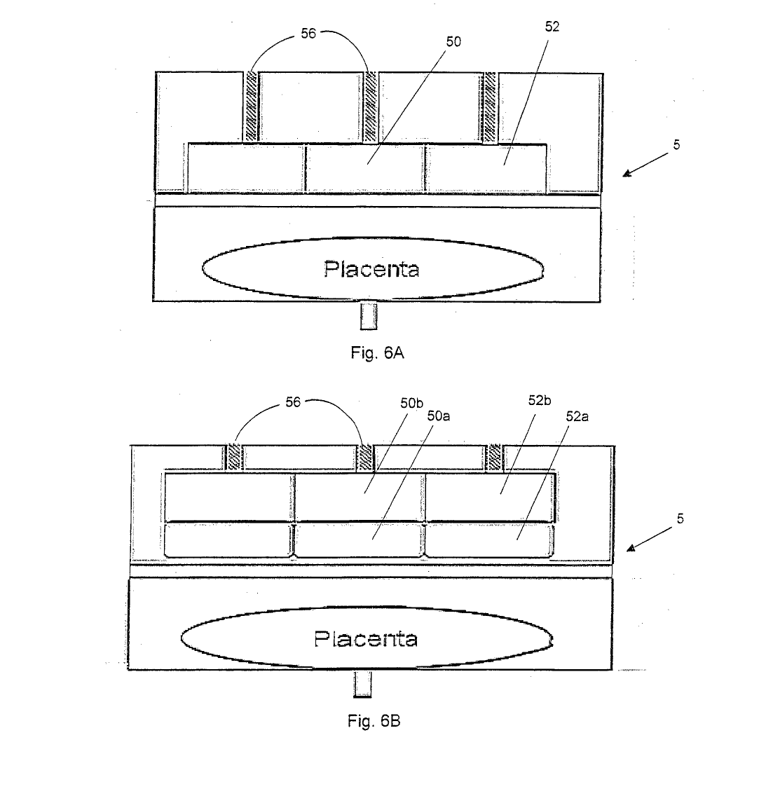

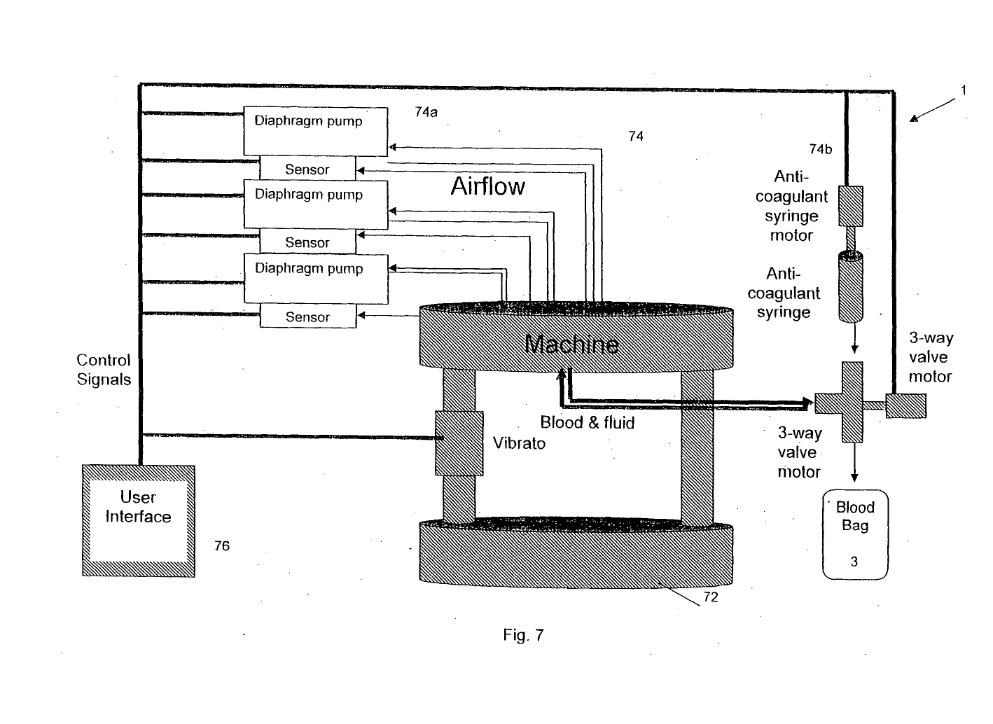

A schematic diagram of a placental blood extracting device 1 is shown in FIG. 7. The placental blood extraction device may comprise a main frame 72, a pressure application device 5, perfusion and collection module 74, and control user interface. The frame may include a placenta receiving bay for receiving a receptacle 2 having a placenta therein. The perfusion and collection module 74 may comprise one or more mechanical pumps 74a, perfusion means 74b, perfusant solution application means 74c, so as to apply the perfusion during blood extraction, with the perfusant solution to disassociate the hematopoietic progenitor cells in the placenta and thereby assisting the blood extraction. For example, the perfusant may be an anti-coagulant. The main frame 72 may include a housing with a side opening, through with the receptacle 2 may be inserted into the placenta receiving bay. The top of the frame houses the pressure application device. The pressure application device 5 applies pressure onto the placenta inside the receptacle 2 through the cover 24.

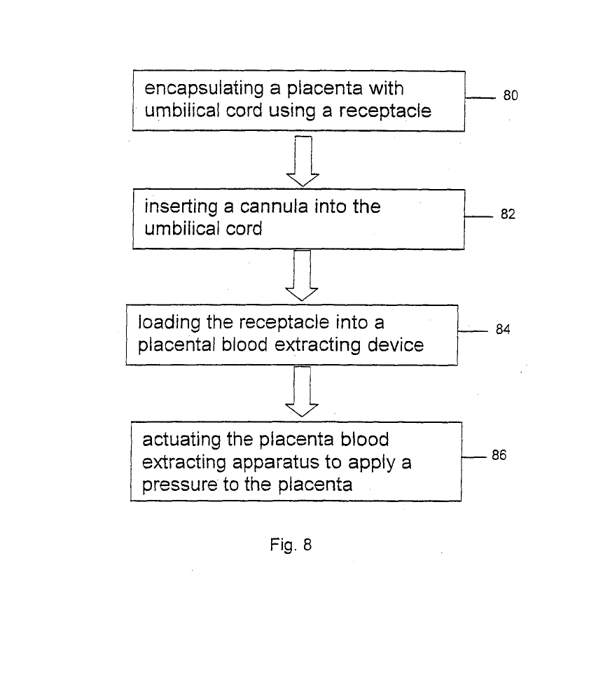

A flow diagram of the placental blood extracting process is shown in FIG. 8. The process includes: encapsulating 80 the placenta with umbilical cord using a receptacle, inserting 82 a cannula into the umbilical cord, loading 84 the receptacle into a placental blood extracting device, actuating 86 the placenta blood extracting apparatus to apply a pressure to the placenta, and collecting blood flowing from the placenta into the cannula.

The placenta is first prepared by encapsulating the placenta using a receptacle 2. The process may further include a step of loading the empty receptacle into a placental blood extracting device prior to encapsulation. The encapsulation may include clipping an end of the umbilical cord using a weight and allowing the end to fall into the tube 22. The placenta enclosed in the compartment 20 and the umbilical cord enclosed in the tube are exposed and disinfected.

Subsequently, the umbilical cord is cannulated 84 with the catheter needle. The process may further include a step of perfusing a medium into the cord and placenta. The medium could include aqueous solutions, anti-coagulant and/or chemicals which aid content retrieval from the placenta and cord. In one embodiment, the placenta is perfused with a flushing solution containing anti-coagulant. Perfusion is aided by a mechanical pump. Upon perfusion, the hematopoietic progenitors present in the placenta and the cord are dissociated from the surrounding tissue.

The receptacle 2 with a placenta therein may then be loaded into the placenta receiving bay of the placental blood extracting device 1. In one embodiment, the receptacle may be in sliding engagement with the device, such that it slides on a rail to receive the placenta. Further, the receptacle may also be in rotational engagement, such that the receptacle is inverted to allow placement of the placenta maternal side first, then reverted back to the normal position. The receptacle can then be slid back into the device, following cannulation.

Lastly, the placenta blood extracting apparatus is actuated to apply a pressure to the placenta. The cellular extraction is aided by the application of pressure on the maternal surface of the placenta via an air bag system. Applied pressure is monitored through the control user interface 76 so as to prevent the excessive pressurization. Subsequently, cord blood is drained via the tubes into sterile blood bags. This actuating step may include applying vibration to the placenta. The process may further comprise a step of applying a negative pressure to the cannula to facilitate the collection of blood.

In a further embodiment, in order to improve yield, the device may include an arrangement whereby fluid is injected into the placenta in order to apply additional pressure for the extraction of blood. To this end, the fluid path created by such an arrangement of the device may include:
i) An injection of fluid through a cannula placed in an umbilical vein, so as to perfuse fluid through the veins of the placenta;
ii) The injection of fluid builds pressure in the placenta, which tends the evacuate blood from the placenta into the cord;
iii) The evacuated blood is then extracted through a second cannula in the cord.

Alternatives to this fluid path include having the injection cannula and extraction cannula being the same, with a 3 way valve to accommodate the injection and extraction functions.

The perfused fluid may include anti-coagulant, saline solution or other flushing agent.

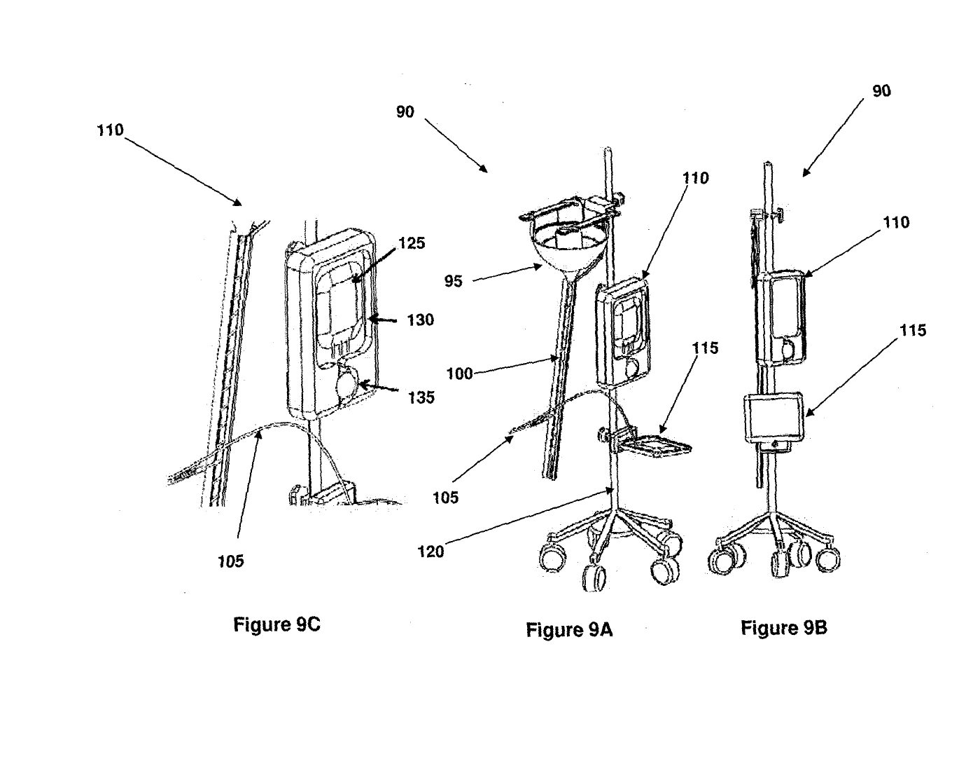

FIGS. 9A and 9B shows the placental blood extraction device according to one embodiment of the present invention, in this case a placenta transfer system 90. The system 90 includes a receptacle 95, 100 for receiving the placenta 95 and umbilical cord 100 so as to allow a catheter and conduit, or tube, 105 to extract the cord blood and deliver to a blood bag located on a blood bag rocker 115. The system further includes a perfusion system 110 shown in more detail in FIG. 9C. The system, in this case a perfusion device 110 includes a perfusate bag 125 which fits within the casing of the device 110. The device 110 further includes a warming device 130 for maintaining the perfusate at a predetermined temperature so as to ease blood flow. By way of example, the warmer 130 could maintain the perfusate in a range 35° C. to 40° C. or more specifically it may warm the perfusate to 37° C. The warming device may be applied to the bag of perfusant, such as a bag of anti-coagulant, or may be applied to the conduit, or tubing, used to transfer the perfusant.

The device 110 further includes a peristaltic pump 135 so as to pump the perfusate into the placenta for maintaining a back pressure as well adding perfusate to the cord blood for ease of collection. Whilst a peristaltic pump is identified, the skilled person will appreciate other forms of pressure actuation may be used, including a syringe pump. To this end, the perfusion may be stored in said syringe pump replacing the need for a bag.

During in utero collection, once the baby is delivered, the umbilical cord is severed and cannulated so as to attach the collection conduit/tube 105 to the umbilical cord. When the placenta is delivered it is encapsulated within the receptacle 95, 100 which may be secured to a platform for ex utero extraction. Pre-warmed perfusate will be perfused into the placenta using the peristaltic pump 135 and drained out of the placenta through gravity. From the system 90 the receptacle 95, 100 containing the placenta may be transferred to a separate machine so as to allow pressure to be applied to the placenta for further cord blood extraction. Alternatively, the pressure applicator may be mounted to the system 90 to apply pressure to the placenta whilst mounted to the system 90. Once the cord blood collection is complete, the system 90 can be folded (FIG. 9B) and placed in a more convenient location having a smaller footprint consistent with the crowded delivery suite.

The system is arranged to reduce the turnaround time between the in utero and ex utero cord blood collection by having the necessary components integrated into a single system 90.

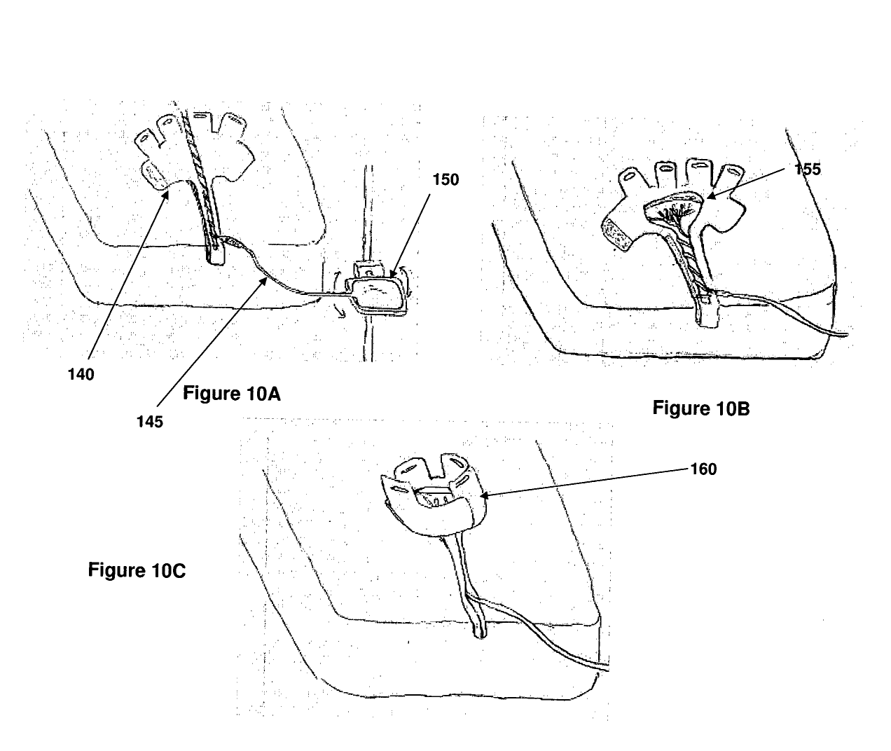

FIG. 10A to 10C show a sequential view of the transition from in utero to ex utero cord blood collection. While the placenta 155 is in the uterus the umbilical cord is placed on an absorbent receptacle 140. The cord is wiped dry and then cannulated so as to connect the cord to a blood bag located on a blood bag rocker 150 through a tube 145. After the placenta 155 is delivered it is placed on the receptacle 140 which then wraps 160 around the placenta and secured using Velcro. As an alternative to Velcro, buttons, zips or other forms of binding the receptacle about the placenta. The placenta and umbilical cord are then placed into the system as shown in FIG. 9A. In one embodiment, the blood bag rocker may include sensors for determining the volume of blood collected in said bag. For instance, a load cell may measure the weight of the bag, and connected to a display for communicating the weight, blood volume or % of bag volume to highlight to the healthcare worker the collected volume, such as to replace the bag or merely to provide information.

The system of 9A may be battery powered so as to facilitate portability. Further the system may be mounted on wheels so as to further assist in the easy introduction and removal of the system as required. To achieve the battery powered aspect of the system the batteries may be rechargeable and so connectable to an electrical socket or possibly through induction charging.

The placenta transfer system reduces the critical loss of time during the transition from in utero to ex utero cord blood collection and so minimizing the risk of blood clots and overall improvement of cellular yield. In the embodiment of FIG. 9A the placenta transfer system may be a compact design to allow the process to be conducted within the delivery room without the need of additional manpower transportation or space.

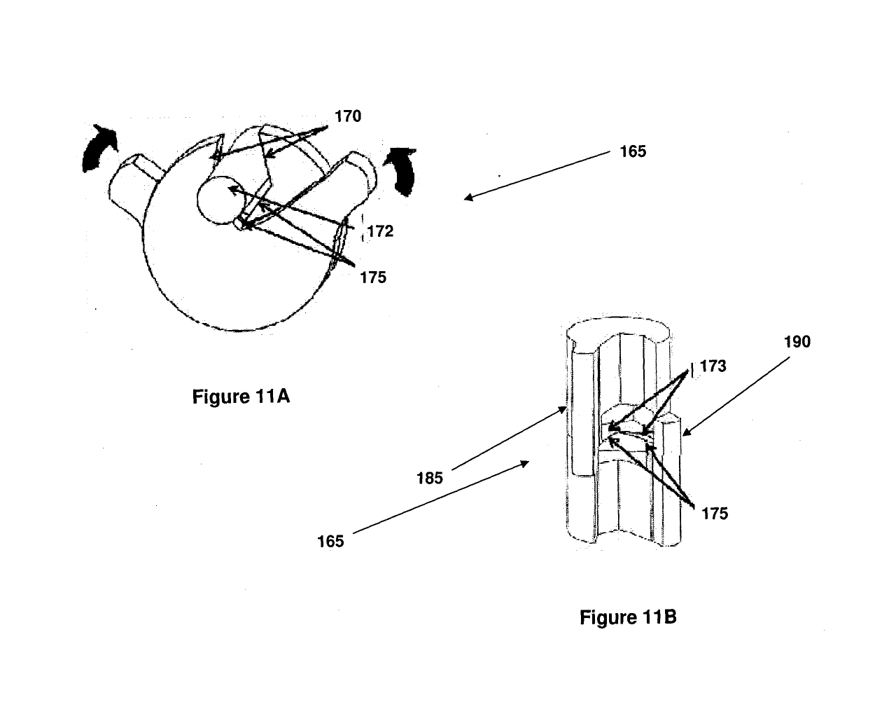

FIGS. 11A and 11B show a cord cutting device 165 arranged to receive and cut an umbilical cord 172. The cord cutting device engages the cord 172 in a manner so that at the time of cutting the open ends of the cord 172 are enclosed within the device and so preventing blood splatter. A further consequence of the enclosure during cutting reduces the risk of injury to healthcare workers by both cutting the cord within the device and also providing a secured grip of the cord during the procedure.

The cord cutting device 165 comprises a first and second portion 185, 190 which are in relative rotational engagement about a common axis. The portions 185, 190 are coupled at respective ends of the portions and have complimentary slots. In the initial position the slots align and so allow the placement of the cord. Mounted to the respective portions include cutting edges 175 at the respective ends. Those areas of the two portions 185, 190 in proximity to the cord 172 act as a barrier to enclose the cord with the cutting edges arranged to then cut the cord on a secondary rotation from the first, initial, position to a second cutting position.

Positioned at the respective ends are clamps, initially placed in an open position. As the cord is placed within the device, it is consequently placed into the open clamps. On first rotating the portions 185, 190 to a third position the clamps close around the cord and lock with sufficient pressure to block blood flow.

The placement of the clamps is such that they block blood loss at the severed ends of the cord following the cord cutting rotation.

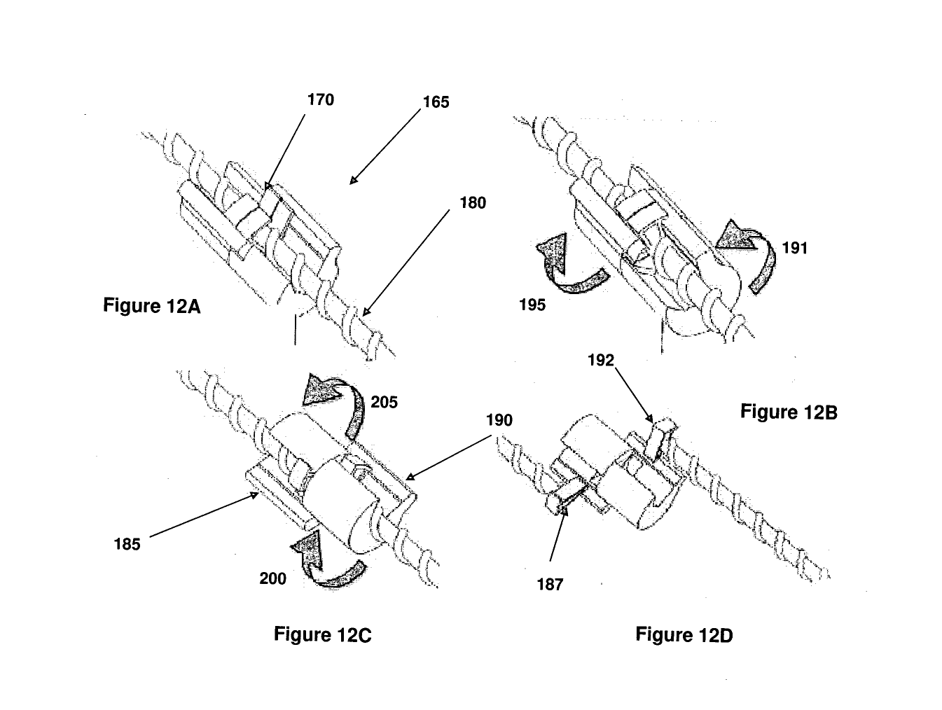

FIGS. 12A to 12D show a sequential view of the cord cutting device 165 whereby the umbilical cord 180 is placed within the aligned slots of the portions of the device 165 in the first position. The two portions 185, 190 are rotated 195, 191 relative to each other to a third position so as to close the device around the cord 180. The two portions 185, 190 are then rotated in an opposed direction 200, 205 which brings the cutting edges into contact with the cord and consequently shearing through. In a further embodiment, the device 165 may include a pair of clamps 187, 192 which as a consequence of the reverse rotation 200, 205 are arranged to clamp the severed ends of the cord 180 so as to close off the cord preventing further loss of cord blood.

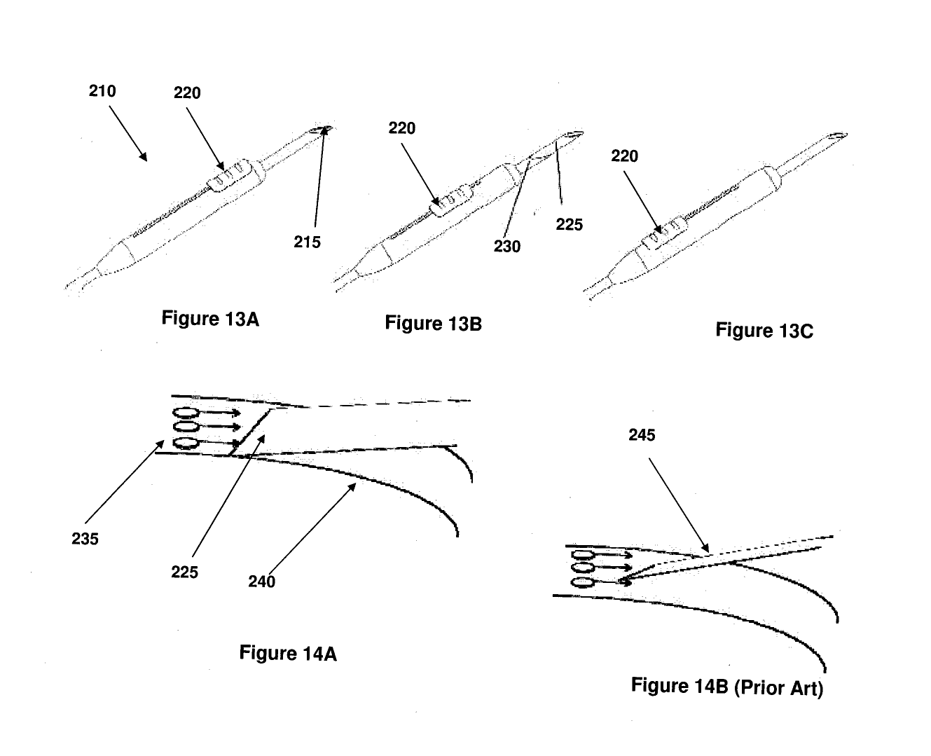

FIGS. 13A to 13C show a cannula assembly 210 according to one embodiment of the present invention. The cannula assembly is applicable in use for most if not all applications of cannula applications. Certain embodiments of the broad invention, such as catheter diameter and the singe handed operation also make the assembly applicable for the extraction of cord blood.

The assembly 210 comprises a pair of concentric sleeves 225, 230 whereby the outer sleeve, being an introducer 230, is arranged for penetrating the umbilical cord so as to place the flexible catheter 225 within the cord vein. The cannula assembly 210 further includes a slide 220 mounted to the outer penetrative sleeve (introducer) 230 and is arranged to retract the penetrative sleeve 230 on insertion into the cord vein leaving the flexible catheter 225 in place. The flexible catheter 225 as shown in FIG. 14A is sized such that the catheter diameter is equal to the diameter of the cord vein 240 so as to more efficiently extract blood 235 from the cord vein 240. FIG. 14B shows a prior art catheter 245 which is typically much smaller than the cord vein 240 and so is not able to efficiently extract cord blood following insertion.

The penetrative sleeve 230 or introducer needle being retractable and lockable in place through use of the slide 220 avoids the disposal of a sharp needle as is the case of prior art cannulae and so prevent stick injuries to healthcare workers. The assembly allows the user to cannulate the cord with one hand and manipulate the cord with the other simultaneously, again unlike the two handed operation of conventional cannulae. The relative operational ease further reduces the time for cannulation and so further prevents the onset of blood clots within the cord as well as more effectively and efficiently extracting blood.

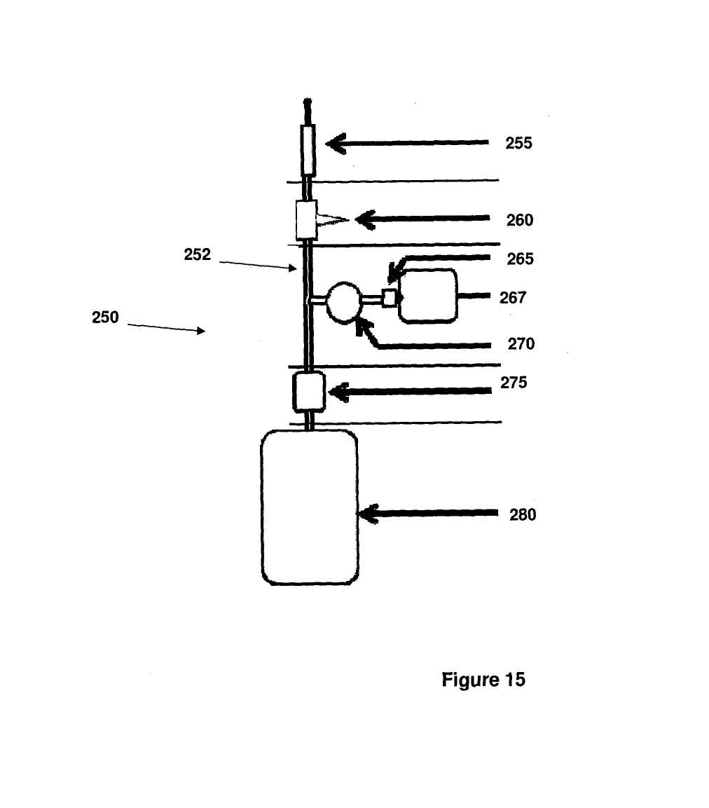

Once the punch is made into the cord and the introducer needle is retracted, the flexible catheter 225 is maneuvered carefully into the cord vein. Unlike prior art cannulae which have a stiff catheter the cannula assembly according to this embodiment uses a soft and flexible catheter designed to fit within the cord vein and preferably apply pressure to the walls of the vein so as to further expand the vein for better cord blood extraction. Ideally, this will capture all the blood flowing through the vein directly into the catheter and certainly considerably greater proportion of the blood then would be available through a catheter of the prior art. Further, the flexible catheter 225 according to the present invention is more fully insertable into the vein and so less chance of the catheter 225 from slipping from the vein during the collection process. FIG. 15 shows a blood bag assembly suitable for use with the placenta transfer system. The blood bag assembly 250 comprises a blood bag 280 having a cannula 255 at an opposed end of a tube system. The cannula 255 may be a conventional cannula or may be a cannula assembly according to one embodiment of the present invention. The assembly 250 includes a blood sampling site 260 to facilitate the extraction of blood for testing. The blood bag assembly 250 further includes a coagulant system comprising an perfusion bag 267 having a spike 265, for selectively mounting the perfusion bag 267, and a peristaltic pump 270.

The connection to the tube 252 allows for selective use of the perfusion whereby in circumstances where perfusion is not desired or not permitted then the perfusate can be kept away from the flow of cord blood to the blood bag 280. The peristaltic pump may be selectively controllable so as to control the volume of perfusate that is pumped into the cord. A blood sampling site 260 is also included to allow healthcare workers to withdraw a blood sample for necessary action without interfering with the cord blood collection process. For example, the blood sampling site may be a needleless injection site, vacutainer site or other means for gaining access to the flow of cord blood without having to interrupt the procedure or blood flow generally.

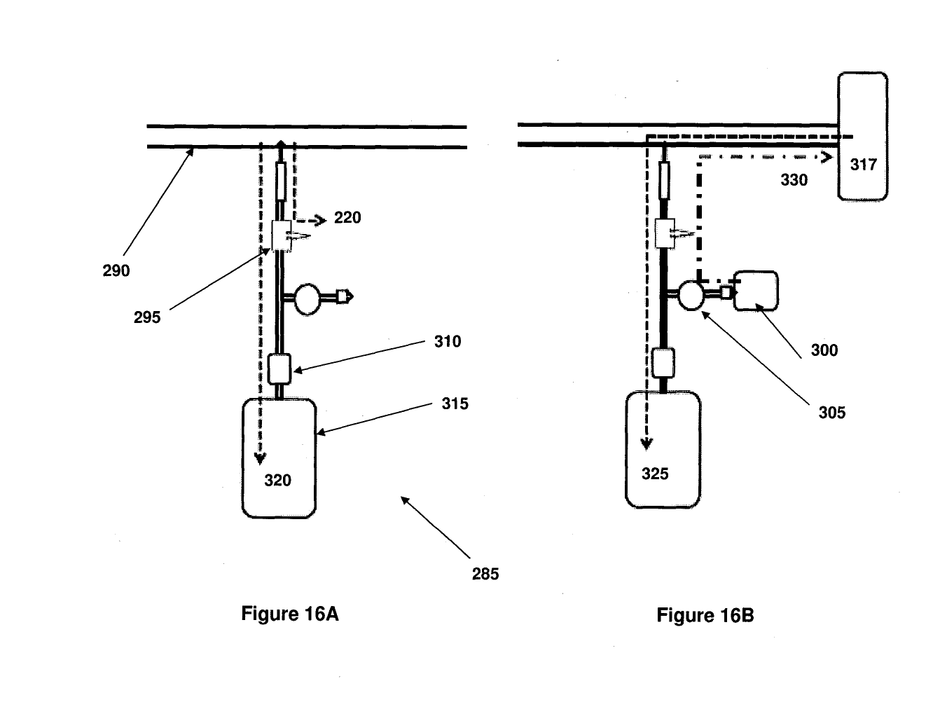

FIG. 16A shows the in utero collection process using a cord blood bag assembly 285. Here the umbilical cord 290 has a cannula inserted therein during the period prior to delivery of the placenta. Cord blood is collected for testing 220 through the blood sampling site 295. Cord blood is then collected 320 in the blood bag 315. Once the placenta has been delivered the arrangement shown in FIG. 16B whereby the delivered placenta 317 becomes the source of cord blood collection 325 into the bag 315. To facilitate the collection process the blood bag assembly 285 may include a perfusion bag 300 and peristaltic pump 305 for pumping 330 perfusate into the placenta 317.

1. A placental blood extraction device comprising:
a receptacle for receiving a placenta with umbilical cord, the receptacle comprising
a compartment arranged to receive the placenta; and
a tube extending from the compartment for receiving the umbilical cord;
wherein the receptacle is selectively removable from the device for receiving the placenta.

2. (canceled)

3. (canceled)

4. (canceled)

5. (canceled)

6. (canceled)

7. (canceled)

8. A placental blood extraction device comprising:
a pressure application device for applying pressure to a placenta, the pressure application device comprising a plurality of pressing members for applying pressure on a plurality of regions of the placenta.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A cannula assembly comprising:
a catheter;
a housing mounted to a portion of said catheter such that a length of catheter projects from said housing;
an introducer in sliding engagement with said housing and arranged coaxially with said catheter
wherein the introducer is movable from an extended position having a penetration end contiguous with an end of said catheter and a retracted position such that said catheter end projects from said penetration end.

40. The cannula assembly according to claim 39, further including a selectively releasable lock arranged to lock the introducer in the retracted position.

41. The cannula assembly according to claim 39 or 40, wherein the introducer is a concentric sleeve positioned externally to the catheter.

42. The cannula assembly according to claim 39, wherein the retracted position places the introducer fully retractable within the housing.

43. The cannula assembly according to claim 39, wherein the assembly is arranged for penetrating a vein of an umbilical cord, said catheter having a diameter equal to a diameter of the cord vein.

44. A blood bag assembly comprising:
a blood transfer conduit;
a blood bag coupled at a first end of said conduit;
a cannula coupled at an opposed end of said conduit;
a blood sampling site for obtaining a blood sample coupled at a position intermediate said ends;
a tapping for an perfusion system coupled at a position intermediate said ends.

45. (canceled)

46. (canceled)

47. (canceled)

48. (canceled)

49. (canceled)

50. The cannula assembly according to claim 39, further including a slide arranged to single handedly project said introduced from the housing.

51. The cannula assembly according to claim 39, wherein the catheter of the cannula assembly is soft and flexible.

52. The cannula assembly according to claim 51, wherein the flexible catheter is arranged to expand the vein.

53. The cannula assembly according to claim 52, wherein the flexible catheter is arranged to engage with the expanded vein to reduce slipping between the vein and catheter.

54. The cannula assembly according to claim 52, wherein the flexible catheter is arranged to engage with the expanded vein to such that a diameter of the catheter is equal to a diameter of the cord vein.

55. The cannula assembly according to claim 39, further including a blood bag assembly coupled to said catheter with a conduit intermediate the blood bag assembly and the catheter.

56. The cannula assembly according to claim 55, wherein the blood bag assembly includes a 3 way junction, for controllable fluid pathways amongst the 3 ports on the junction;
one of said ports including:
a blood sampling site for obtaining a blood sample coupled at a position intermediate said ends;
a tapping for an perfusion system coupled at a position intermediate said ends.
a syringe port coupled at a position intermediate said ends;
a port arranged to apply negative pressure to the cannula coupled at a position intermediate said ends.

57. The cannula assembly according to claim 55 or 56, wherein the blood bag assembly includes a perfusion system coupled to the perfusate tapping.

58. The cannula assembly according to claim 57, wherein the perfusion system includes a perfusate bag.

59. The cannula assembly according to claim 57 or 58, wherein the perfusion system includes a tapping for a peristaltic pump.

60. The cannula assembly according to claim 57, wherein the perfusion system includes a warming device for warming the perfusate bag and/or the conduit used to transfer the perfusate.

61. The cannula assembly according to claim 57, wherein the retractable introducer concentrically and externally placed about the catheter permits application of the cannula assembly to the placenta in-utero.
